(19) United States
(12) Patent Application Publication
(10) Pub. No.: US 2011/0151486 A1
(43) Pub. Date: Jun. 23, 2011

(54) METHODS AND SYSTEMS TO PREVENT GAS BUBBLES FROM INTERFERING WITH FLOW OF FLUID THROUGH A MEMBRANE REGION

(75) Inventors: Thomas M. Zappia, Somerville, MD (US); Brandon T. Johnson, Cambridge, MA (US); David Butz, Groton, MA (US)

(73) Assignee: Boston Microfluidics, Cambridge, MA (US)

(21) Appl. No.: 12/908,803

(22) Filed: Oct. 20, 2010

Related U.S. Application Data
(63) Continuation-in-part of application No. 12/228,081, filed on Jul. 16, 2008.

(51) Int. Cl. G01N 33/53; C12M 1/34

(52) U.S. Cl. 435/7.92; 435/287.2

(57) ABSTRACT

Methods and systems to remove gas bubbles from liquids and to improve uniform fluid flow through a region of a membrane in a microfluidic device, including to reduce, remove, and/or prevent gas bubbles on a surface of a porous membrane. An example membrane bubble trap system may include a fluid channel connected to a bubble pathway that surrounds an opening sealed with a membrane. The bubble pathway may be configured to collect bubbles in fluid that passes through the membrane through buoyancy forces and through a directional feature of a curved surface placed above the membrane.

### CROSS REFERENCE

This application is a continuation-in-part of U.S. Utility patent application Ser. No. 12/228,081, filed Jul. 16, 2008, and claims the benefit of:

U.S. Provisional Application No. 61/253,356, filed Oct. 20, 2009;

U.S. Provisional Application No. 61/253,365, filed Oct. 20, 2009;

U.S. Provisional Application No. 61/253,373, filed Oct. 20, 2009;

U.S. Provisional Application No. 61/253,377, filed Oct. 20, 2009;

U.S. Provisional Application No. 61/253,383, filed Oct. 20, 2009; and

U.S. Provisional Application No. 61/266,019, filed Dec. 2, 2009;

all of which are incorporated herein by reference in their entireties.

### TECHNICAL FIELD

Disclosed herein are methods and systems to capture or trap gas bubbles in liquids, such as to improve uniformity of fluid flow through a region of a membrane in a microfluidic device.

### BACKGROUND

When a liquid fluid flows through, or is forced through a membrane, gas bubbles within the liquid may collect on a surface of the membrane and may interfere with liquid flow through the membrane.

In an assay system, a membrane, such as a nitrous cellulose based membrane, may be used in combination with a fluid sample to detect the possible presence of a chemical or biological target in a sample. These membranes provide support and large binding capacity for immobilizing markers that will indicate the presence of chemical or biological targets, such as taught in U.S. Pat. No. 4,066,512 (Biologically active membrane material, Chung Jung Lai et al). An example of a process that uses these membranes is an enzyme-linked immunosorbent assay (ELISA).

In a lab, the ELISA process is usually carried in a microtiter plate. The membrane is placed in the bottom of the plate and various fluids are washed over the membrane. Test that are run outside the lab require the chemistry and sample to be applied to a self contained device. In a self contained device, such as a pregnancy test, the reagents needed to carry out the ELISA process are immobilized in different regions of the membrane. Devices such as these began with U.S. Pat. No. 4,999,163 (Disposable, pre-packaged device for conducing immunoassay procedures). These self contained devices use capillary action to move fluid through the membrane. Sample is placed on a collection port and the fluid moves passively through the device as the reaction is carried out. The scale of these devices is too large to have an issue of gas bubbles, unlike microfluidic devices.

Microfluidic devices deal with small volumes. These devices have been developed for the ELISA process because of the benefit of using much smaller amounts of fluid to run the same test traditionally performed in a micro-titer plate. Most of these microfluidic devices are made cheaply out of polystyrene and manufactured by standard lithography techniques. The surfaces of these devices must provide the proper chemistry for immobilization of molecules needed for the ELISA process.

Nitrous cellulose membranes can be combined with microfluidic devices to bring the benefit of a larger reaction area in the membrane with the smaller volume use of the microfluidic device. Most microfluidic devices still use capillary forces to move fluid, while some can be assembled so fluid flow is pushed through a region of the membrane, either by gravity or centrifugal forces.

When fluid contacts the membrane, it is generally retained in pores of the material by surface tension and capillary forces. Certain pressure is required to overcome these forces and push more fluid or gas through the membrane. It has been observed that there is less resistance to push fluid rather than gas through a wet membrane. This imbalance causes a gas bubble trapped on the surface of the membrane to interfere with the fluid flow through that section of the membrane. If uniform fluid flow through that section is required—for example to evenly deposit material contained in the fluid on that membrane or to ensure that material embedded in that membrane has full contact with the fluid—it will not be achieved if a gas bubble is trapped on the surface, as fluid will flow around the gas bubble and not come in contact with the membrane directly underneath it. If the membrane is performing the ELISA process, this can lead to a significant reduction of signal as the fluid sample or reagents cannot fully contact the membrane.

These gas bubbles can form when fluid with gas travels to a termination region blocked by a membrane. The gas bubbles must either be forced through the membrane or stay in the termination region. As fluid channels are reduced to ever smaller dimensions, a need for effective gas bubble blocking increases.

To be most effective, microfluidic devices with fluid and gas flow need to deal with the problems created by the presence of interfering gas bubbles. A number of techniques have been tried to mitigate bubble formation and bubble entrapment with varying degrees of success. US application 20090123338 to Guan; Xiaosheng (2009) teaches a method to prevent bubble when filling a microfluidic device. European patent EP1792655 teaches a method for trapping bubbles upstream of a predetermined region. Methods like these try to compensate for unknown amounts of gas in a constantly flowing system. Most methods to mitigate bubble formation have been focused on constantly removing the bubbles so they do not interfere with cell cultures or other biological substances that can be affected by gas bubbles.

### SUMMARY

Disclosed herein are methods and systems to capture or trap gas bubbles in fluids, including to trap a predetermined volume of gas bubbles. If the maximum amount of gas needed to trap is known, the system can be designed to work at or below that amount, without the need for complicated vents or active methods to remove gas above a certain amount.

A gas bubble trap may be positioned proximate to an active region of a porous membrane to capture or trap gas bubbles from a liquid fluid that flows through the membrane, and to maintain the trapped gas away from the membrane. The regions of membrane can be considered termination points that gas bubbles can interfere with. Trapping the gas bubbles around the termination points instead of in contact with them prevents fluid contact problems with the membrane region. Relying on buoyancy or centrifugal force, structures can be made to create pathways that collect the gas bubbles, thus directing them into trapping regions instead of active regions that they may interfere with.

Systems and methods to trap gas bubbles, as disclosed herein, may be implemented with self-contained, point-of-care, portable, point-of-care, user-initiated fluidic assay systems. Example assays include diagnostic assays and chemical detection assays. Diagnostic assays include, without limitation, enzyme-linked immuno-sorbent assays (ELISA), and may include one or more sexually transmitted disease (STD) diagnostic assays.

### BRIEF DESCRIPTION OF THE DRAWINGS/FIGURES

In the drawings, like reference numbers indicate identical or functionally similar elements. Additionally, the leftmost digit(s) of a reference number identifies the drawing in which the reference number first appears.

FIG. 1 is a process flowchart of a method of performing an assay with a substantially self-contained, point-of-care, user-initiated fluidic assay system.

FIG. 2 is a block diagram of a portable, point-of-care, user-initiated fluidic assay system.

FIG. 3 is a perspective view of a portable, point-of-care, user-initiated fluidic assay system 300.

FIG. 4 is a process flowchart of a method of preparing a portable, point-of-care, user-initiated fluidic assay system.

FIG. 5 is a process flowchart of a method of using an assay system prepared in accordance with FIG. 4.

FIG. 6 is a perspective view of another assay system 600, including a cover illustrated in a first position.

FIG. 7 is a cross-sectional view of assay system 600, including plungers 702, 704, and 706, wherein the cover is illustrated in the second position.

FIG. 8 is another cross-sectional view of assay system 600, wherein plungers 702, 704, and 706 are in corresponding initial or first positions.

FIG. 9 is another cross-sectional view of assay system 600, wherein plungers 702, 704, and 706 are in respective first intermediate positions.

FIG. 10 is another cross-sectional view of assay system 600, wherein plunger 704 is in a second position, and plungers 702 and 704 are in respective second intermediate positions.

FIG. 11 is another cross-sectional view of assay system 600, wherein plungers 702, 704 and 706 are in respective second positions.

FIG. 12 is an expanded cross-sectional view of a portion of assay system 600, including a portion of plunger 706 in the first position corresponding to FIG. 8.

FIG. 13 is another expanded cross-sectional view of a portion assay system 600, including a portion of plunger 706 in the intermediate position corresponding to FIG. 9.

FIG. 14 is another expanded cross-sectional view of a portion of assay system 600, including a portion of plunger 706 in the second position corresponding to FIGS. 10 and 11.

FIG. 15 is a cross-sectional perspective view of another assay system 1500.

FIG. 16 is a cross-sectional perspective view of another assay system 1600.

FIG. 17 is cross-sectional view of a mechanical actuator system.

FIG. 18 is a profile view of a membrane bubble trap system.

FIG. 19 is a cross-sectional view of the membrane bubble trap system.

FIG. 20 is an upwardly directed view of an upper portion of the membrane bubble trap system.

FIG. 21A through 21C depicts example movement of fluid and gas bubbles through fluid channels and collection of gas bubbles.

FIGS. 22A through 22E are additional cross-sectional views of the membrane bubble trap system, to illustrate fluid flow and bubble trapping.

FIG. 23 is an upwardly directed view of an upper portion of another membrane bubble trap system, including multiple interconnected membrane active areas, each including a corresponding bubble termination trap.

In the drawings, the leftmost digit(s) of a reference number may correspond to the drawing in which the reference number first appears.

### DETAILED DESCRIPTION

Disclosed herein are methods and systems to capture or trap gas bubbles in fluids, including to trap a predetermined volume of gas bubbles.

The methods and systems to trap gas bubbles are described herein with respect to example point-of-care, user-initiated fluidic assay methods and systems, for illustrative purposes. The methods and systems to trap gas bubbles are not, however, limited to the assay methods and systems disclosed herein. Based on the teachings herein, one skilled in the art will understand that the methods and system to trap gas bubbles may be implemented with respect to other assay systems, including diagnostic assays and chemical assays.

An immunoassay is a biochemical test to detect a substance, or measure a concentration of a substance, in a biological sample such as blood, saliva, or urine, using a reaction between an antibody and an antigen specific to the antibody.

An immunoassay may be used to detect the presence of an antigen or an antibody. For example, when detecting an infection, the presence of an antibody against the pathogen may be measured. When detecting hormones such as insulin, the insulin may be used as the antigen.

Accordingly, where a method or system is described herein to detect a primary binding pair molecule using a corresponding second binding pair molecule, it should be understood that the primary binding pair molecule may be an antibody or an antigen, and the second binding pair molecule may be a corresponding antigen or antibody, respectively. Similarly, where a method or system is described herein to detect an antibody or antigen, the method or system may be implemented to detect a corresponding antigen or antibody, respectively.

Immunoassays may also be used to detect potential food allergens and chemicals, or drugs.

Immunoassays include labeled immunoassays to provide a visual indication of a binding pair of molecules. Labeling may include an enzyme, radioisotopes, magnetic labels, fluorescence, agglutination, nephelometry, turbidimetry and western blot.

Labeled immunoassays include competitive and non-competitive immunoassays. In a competitive immunoassay, an antigen in a sample competes with labeled antigen to bind with antibodies. The amount of labeled antigen bound to the antibody site is inversely proportional to the concentration of antigen in the sample. In noncompetitive immunoassays, also referred to as sandwich assays, antigen in a sample is bound to an antibody site. The labeled antibody is then bound to the antigen. The amount of labeled antibody on the site is directly proportional to the concentration of the antigen in the sample.

Labeled immunoassays include enzyme-linked immuno-sorbent assays (ELISA).

In an example immunoassay, a biological sample is tested for a presence of a primary binding pair molecule. A corresponding binding pair molecule that is specific to the primary binding pair molecule is immobilized on an assay substrate. The biological sample is contacted to the assay substrate. Any primary binding pair molecules in the biological sample attach to, or are captured by the corresponding binding pair molecules. The primary binding pair molecules are also contacted with labeled secondary binding pair molecules that attach to the primary binding pair molecules. This may be performed subsequent to, prior to, or simultaneously with the contacting of the primary binding pair molecule with the corresponding immobilized binding pair molecule. Un-reacted components of the biological sample and fluids may be removed, or washed from the assay substrate. Presence of the label on the assay substrate indicates the presence of the primary binding pair molecule in the biological sample.

The label may include a directly detectable label, which may be visible to a human observer, such as gold particles in a colloid or solution, commonly referred to as colloidal gold.

The label may include an indirect label, such an enzyme whereby the enzyme works on a substrate to produce a detectable reaction product. For example, an enzyme may attach to the primary binding pair molecule, and a substance that the enzyme converts to a detectable signal, such as a fluorescence signal, is contacted to the assay substrate. When light is directed at the assay substrate, any binding pair molecule complexes will fluoresce so that the presence of the primary binding pair molecule is observable.

An immunoassay may utilize one or more fluid solutions, which may include a dilutent solution to fluidize the biological sample, a conjugate solution having the labeled secondary binding pair molecules, and one or more wash solutions. The biological sample and fluids may be brought into contact, concurrently or sequentially with the assay substrate. The assay substrate may include an assay surface or an assay membrane, prepared with a coating of the corresponding binding pair molecules.

As described above, the second binding pair molecules may include an antigen that is specific to an antibody to be detected in a biological sample, or may include antibody that is specific to an antigen to be detected in the biological sample. By way of illustration, if the primary binding pair molecule to be detected is an antigen, the immobilized binding pair molecule and the secondary labeled binding pair molecule will be antibodies, both of which react with the antigen. When the antigen is present in the biological sample, the antigen will be immobilized by the immobilized antibody and labeled by the labeled secondary antibody, to form a sandwich-like construction, or complex.

It is known that non-specific or un-reacted components may be beneficially removed using wash solutions, often between processes and/or prior to a label detection process, in order to improve sensitivity and signal-to-noise ratios of the assay. Other permutations are possible as well. For example, a conjugate solution, such as a labeled secondary binding pair molecule solution may be mixed with or act as a sample dilutent to advantageously transport the biological sample to the assay substrate, to permit simultaneous binding of the primary binding pair molecule and the labeled secondary binding pair molecule to the immobilized binding pair molecule. Alternatively, or additionally, the sample dilutent may include one or more detergents and/or lysing agents to advantageously reduce deleterious effects of other components of the biological sample such as cellular membranes, non-useful cells like erythrocytes and the like.

Those skilled in the art will readily recognize that such fluid components and the order of the reactionary steps may be readily adjusted along with concentrations of the respective components in order to optimize detection or distinguishment of analytes, increase sensitivity, reduce non-specific reactions, and improve signal to noise ratios.

As will be readily understood, if the secondary antibody is labeled with an enzyme instead of a fluorescent or other immediately detectable label, an additional substrate may be utilized to allow the enzyme to produce a reaction product which will be advantageously detectable. An advantage of using an enzyme based label is that the detectable signal may increase over time as the enzyme works on an excess of substrate to produce a detectable product.

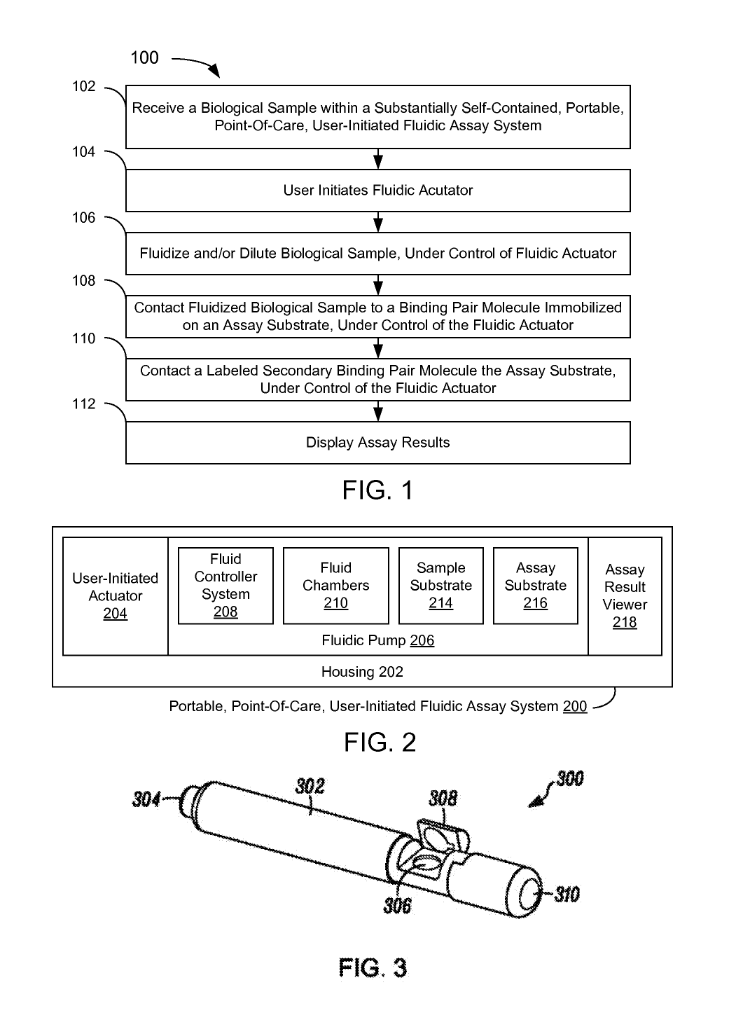

FIG. 1 is a process flowchart of a method 100 of detecting a primary binding pair molecule in a biological sample, using a substantially self-contained, point-of-care, user-initiated fluidic assay system. The primary binding pair molecule may correspond to an antibody or an antigen.

At 102, a biological sample is provided to the assay system. The biological sample may include one or more of a blood sample, a saliva sample, and a urine sample. The biological sample may be applied to a sample substrate within the assay system.

At 104, a fluidic actuator within the assay system is initiated by a user. The fluidic actuator may include a mechanical actuator, such as a compressed spring actuator, and may be initiated with a button, switch, or lever. The fluidic actuator may be configured to impart one or more of a physical force, pressure, centripetal force, gas pressure, gravitational force, and combinations thereof, on a fluid controller system within the assay system.

At 106, the biological sample is fluidized with a dilutent fluid. The dilutent fluid may flow over or through the sample substrate, under control of the fluid controller system.

At 108, the fluidized biological sample is contacted to a corresponding binding pair molecule that is specific to primary binding pair molecule. The corresponding binding pair molecule may be immobilized on an assay substrate within the assay system. The fluidized biological sample may flow over or through the assay substrate, under control of the fluid controller system.

Where the fluidized biological sample includes the primary binding pair molecule, the primary binding pair molecule attaches to the corresponding binding pair molecule and becomes immobilized on the assay substrate. For example, where the second binding pair molecule includes a portion of a pathogen, and where the biological sample includes an antibody to the pathogen, the antibody attaches to the antigen immobilized at the assay substrate.

At 110, a labeled conjugate solution is contacted to the assay substrate, under control of the fluid controller system. The labeled conjugate solution includes a secondary binding pair molecule to bind with the primary binding pair molecule. Where the primary binding pair molecule is immobilized on the assay substrate with the corresponding binding pair molecule, the secondary binding pair molecule attaches to the immobilized primary binding pair molecule, effectively creating a sandwich-like construct of the primary binding pair molecule, the corresponding binding pair molecule, and the labeled secondary binding pair molecule.

The secondary binding pair molecule may be selected as one that targets one or more proteins commonly found in the biological sample. For example, where the biological sample includes a human blood sample, the secondary binding pair molecule may include an antibody generated by a non-human animal in response to the one or more proteins commonly found in human blood.

The secondary binding pair molecule may be labeled with human-visible particles, such as a gold colloid, or suspension of gold particles in a fluid such as water. Alternatively, or additionally, the secondary binding pair molecule may be labeled with a fluorescent probe.

Where the labeled secondary binding pair molecule attaches to a primary binding pair molecule that is attached to a corresponding binding pair molecule, at 110, the label is viewable by the user at 112.

Method 100 may be implemented to perform multiple diagnostic assays in an assay system. For example, a plurality of antigens, each specific to a different antibody, may be immobilized on one or more assay substrates within an assay system. Similarly, a plurality of antibodies, each specific to a different antigen, may be immobilized on one or more assay substrates within an assay system

FIG. 2 is a block diagram of a portable, point-of-care, user-initiated fluidic assay system 200, including a housing 202, a user-initiated actuator 204, a fluidic pump 206, and an assay result viewer 218.

Pump 206 includes one or more fluid chambers 210, to contain fluids to be used in an assay. One or more of fluid chambers 210 may have, without limitation, a volume in a range of 0.5 to 2 milliliters.

Pump 206 includes a sample substrate 214 to hold a sample. Sample substrate 214 may include a surface or a membrane positioned within a cavity or a chamber of housing 202, to receive one or more samples, as described above.

Sample substrate 214 may include a porous and/or absorptive material, which may be configured to absorb a volume of liquid in a range of 10 to 500 μL, including within a range of up to 200 μL, and including a range of approximately 25 to 50 μL.

Pump 206 includes an assay substrate 216 to hold an assay material. Assay substrate 216 may include a surface or a membrane positioned within a cavity or chamber of housing 202, to receive one or more assay compounds or biological components, such as an antigen or an antibody, as described above.

Fluid chambers 210 may include a waste fluid chamber.

Pump 206 further includes a fluid controller system 208, which may include a plurality of fluid controllers, to control fluid flow from one or more fluid chambers 212 to one or more of sample substrate 214 and assay substrate 216, responsive to actuator 204.

Actuator 204 may include a mechanical actuator, which may include a compressed or compressible spring actuator, and may include a button, switch, lever, twist-activator, or other user-initiated feature.

Assay result viewer 218 may include a display window disposed over an opening through housing 202, over assay substrate 216.

FIG. 3 is a perspective view of an portable, point-of-care, user-initiated fluidic assay system 300, including a housing 302, a user-initiated actuator button 304, a sample substrate 306, and a sample substrate cover 308. Sample substrate cover 308 may be hingedly coupled to housing 302.

Assay system 300 further includes an assay result viewer 310, which may be disposed over an assay substrate. Assay result view 310 may be disposed at an end of assay system 300, as illustrated in FIG. 3, or along a side of assay system 300.

Assay system 300 may have, without limitation, a length in a range of 5 to 8 centimeters and a width of approximately 1 centimeter. Assay system 300 may have a substantially cylindrical shape, as illustrated in FIG. 3, or other shape.

Assay system 300, or portions thereof, may be implemented with one or more substantially rigid materials, and/or with one or more flexible or pliable materials, including, without limitation, polypropylene.

Example portable, point-of-care, user-initiated fluidic assay systems are disclosed further below.

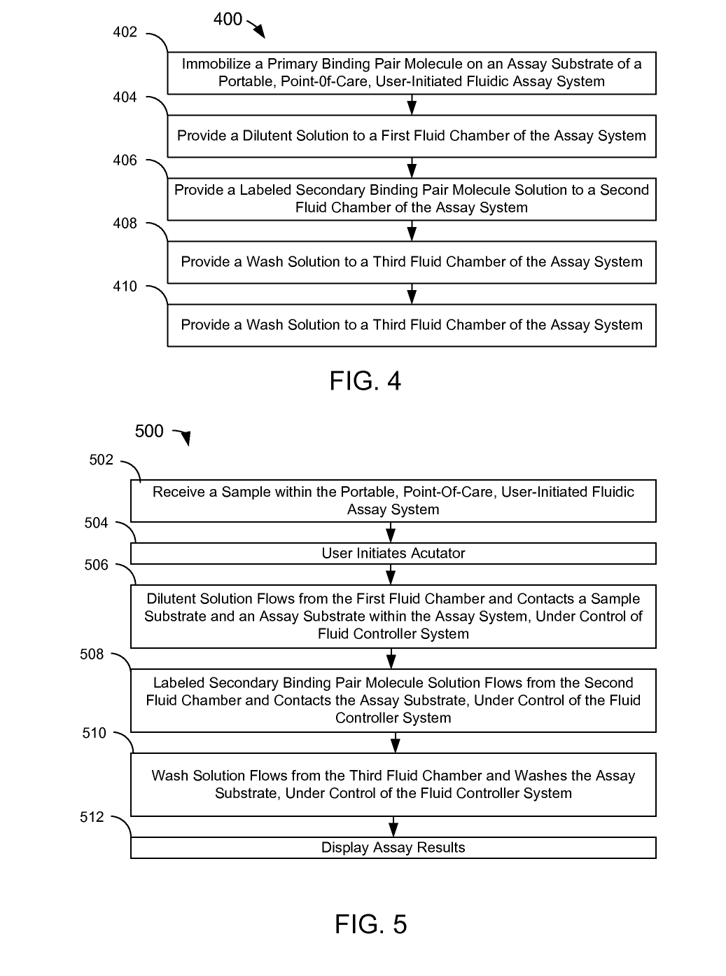

FIG. 4 is a process flowchart of a method 400 of preparing a portable, point-of-care, user-initiated fluidic assay system. Method 400 is described below with reference to assay system 200 in FIG. 2, for illustrative purposes. Method 400 is not, however, limited to the example of FIG. 2.

At 402, a binding pair molecule is immobilized on an assay substrate, such as assay substrate 216 in FIG. 2. The binding pair molecule may include an antigen specific to an antibody, or an antibody specific to an antigen.

At 404, a first one of fluid chambers 210 is provided with a dilutent solution to fluidize a sample.

At 406, a second one of fluid chambers 210 is provided with a labeled secondary binding pair molecule solution.

At 408, a third one of fluid chambers 210 is provided with a wash solution, which may include one or more of a saline solution and a detergent. The wash solution may be substantially similar to the dilutent solution.

FIG. 5 is a process flowchart of a method 500 of using an assay system prepared in accordance with method 400. Method 500 is described below with reference to assay system 200 in FIG. 2, and assay system 300 in FIG. 3, for illustrative purposes. Method 500 is not, however, limited to the examples of FIG. 2 and FIG. 3.

At 502, a sample is provided to a sample substrate, such as sample substrate 214 in FIG. 2, and sample substrate 306 in FIG. 3.

At 504, a user-initiated actuator is initiated by the user, such as user-initiated activator 204 in FIG. 2, and button 304 in FIG. 3. The user initiated actuator acts upon a fluid controller system, such as fluid controller system 208 in FIG. 2.

At 506, the dilutent solution flows from first fluid chamber and contacts the sample substrate and the assay substrate, under control of the fluid controller system.

As the dilutent fluid flows over or through the sample substrate, the sample is dislodged from the sample substrate and flows with the dilutent solution to the assay substrate.

At 508, the labeled secondary binding pair solution flows from the second fluid chamber and contacts the assay substrate, under control of the fluid controller system. The labeled secondary binding pair solution may flow directly to the assay substrate or may flow over or through the sample substrate.

At 510, the wash solution flows from the third fluid chamber and washes the assay substrate, under control of fluid controller system 208. The wash solution may flow from the assay substrate to a waste fluid chamber,

At 512, assay results are viewable, such as at assay result viewer 218 in FIG. 2, and assay result viewer 310 in FIG. 3.

An assay substrate may include a nitrocellulose-based membrane, available from Invitrogen Corporatation, of Carlsbad, Calif.

Preparation of a nitrocellulose-based membrane may include incubation for approximately thirty (30) minutes in a solution of 0.2 mg/mL protein A, available from Sigma-Aldrich Corporation, of St. Louis, Mo., in a phosphate buffered saline solution (PBS), and then dried at approximately 37° for approximately fifteen (15) minutes. 1 μL of PBS may be added to the dry membrane and allowed to dry at room temperature. Alternatively, 1 μL of an N-Hydroxysuccinimide (NHS) solution, available from Sigma-Aldrich Corporation, of St. Louis, Mo., may be added to the dry membrane and allowed to dry at room temperature.

An assay method and/or system may utilize or include approximately 100 μL of PBS/0.05% Tween wash buffer, available from Sigma-Aldrich Corporation, of St. Louis, Mo., and may utilize or include approximately 100 μL of protein G colloidal gold, available from Pierce Corporation, of Rockland, Ill.

An assay method and/or system may be configured to test for Chlamydia, and may utilize or include a sample membrane treated with wheat germ agglutinin, to which an approximately 50 μL blood sample is applied. Approximately 150 μL of a lysing solution may then be passed through the sample membrane and then contacted to an assay substrate. Thereafter, approximately 100 μL of a colloidal gold solution may be contacted to the assay substrate. Thereafter, approximately 500 μL of a wash solution, which may include the lysing solution, may be contacted to the assay membrane without passing through the sample membrane.

Additional example assay features and embodiments are disclosed below. Based on the description herein, one skilled in the relevant art(s) will understand that features and embodiments described herein may be practiced in various combinations with one another.

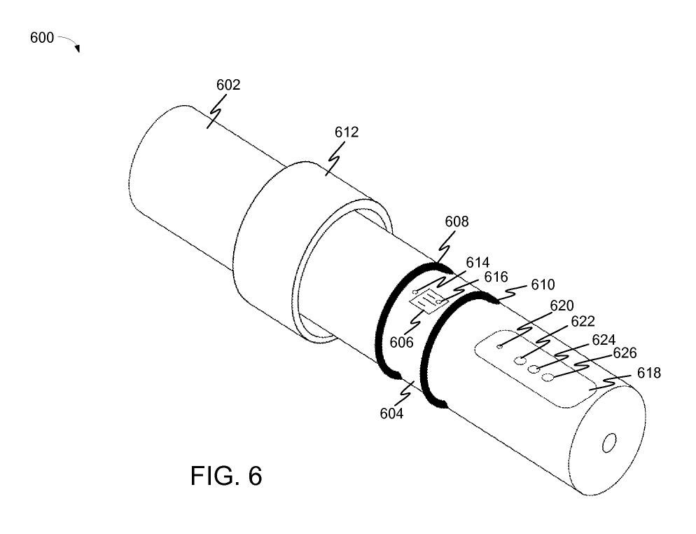

FIG. 6 is a perspective view of an assay system 600, including a body 602 having a sample collection region 604 to receive a sample collection pad or membrane 606, which may include a porous material such as, for example, a glass fiber pad, to absorb a fluid sample.

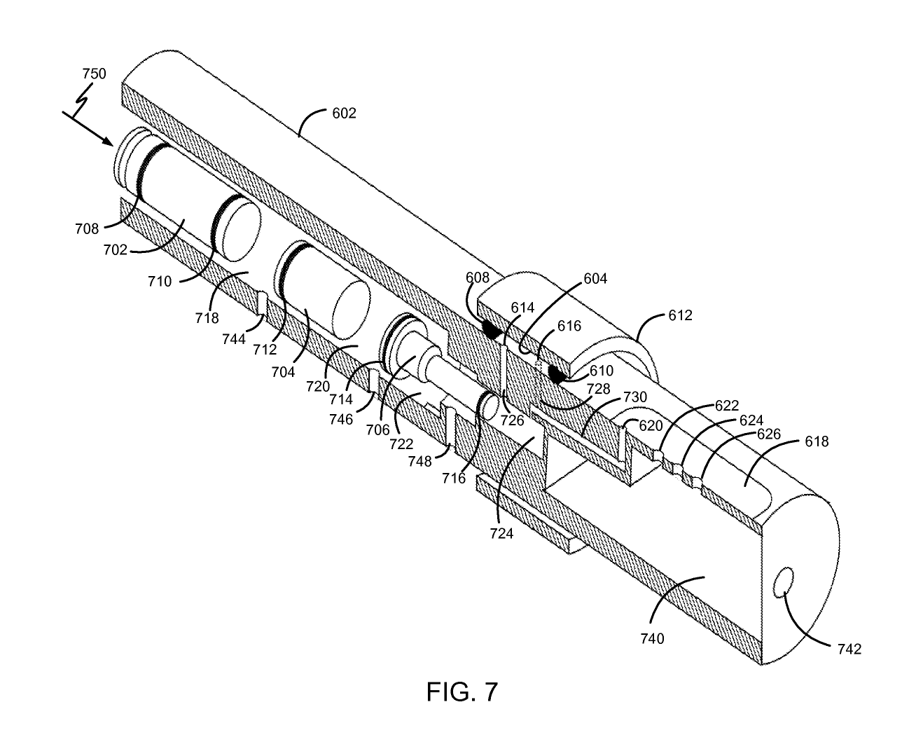

In the example of FIG. 6, sample collection region 604 is positioned between first and second O-rings 608 and 610, and system 600 includes a cover 612 slideably moveable relative to body 602, between a first position illustrated in FIG. 6, and a second position described below with reference to FIG. 7.

FIG. 7 is a cross-sectional view of assay system 600, wherein cover 612 is illustrated in the second position, and sample collection region 604 is bounded by an outer surface of body 602, an inner-surface of cover 612, and O-rings 608 and 610. O-rings 608 and 610 may provide a hermetic seal between sample collection region 604 and an external environment. When cover 612 is in the second position, sample collection region 604 may be referred to as a sample collection chamber.

In FIG. 6, sample collection region 604 includes openings 614 and 616 through the surface of body 602 associated with fluid passages within body 602. Opening 614 may be positioned adjacent to sample collection pad 606, and opening 616 may be positioned beneath sample collection pad 606. System 600 may be configured to provide a fluid through opening 614 into sample collection region 604 and to receive the fluid from sample collection region 604 through opening 616, to cause the fluid to pass through sample collection pad 606.

Body 602 may include an assay region 618 formed or etched within the surface of body 602, having an opening 620 through the surface of body 602 to receive fluid from an associated fluid passage. Assay region 618 may include one or more additional openings to corresponding fluid passages within body 602, illustrated here as openings 622, 624, and 626, to permit the fluid to exit assay region 618.

Assay region 618 may be configured to receive a test membrane having one or more reactive areas, each reactive area positioned on the test membrane in alignment with a corresponding one of openings 622, 624, and 626.

System 600 may include a substantially transparent cover to enclose assay region 618, such as to permit viewing of the test membrane, or portions thereof. The cover may include one or more fluid channels to direct fluid from opening 620 to the membrane areas aligned with openings 622, 624, and 626. Where system 600 includes a cover over assay region 618, assay region 618 may be referred to as an assay chamber.

In FIG. 7, system 600 includes plungers 702, 704, and 706. Plunger 706 is illustrated here as a multi-diameter or stepped plunger. Plunger 702 includes O-rings 708 and 710. Plunger 704 includes an O-ring 712. Plunger 706 includes O-rings 714 and 716. O-rings 708, 710, 712, 714, and 716 may be sized to engage corresponding inner surface portions of body 602. Plungers 702, 704, and 706 are each moveable within body 602 between respective first and second positions and, together with the inner surfaces of body 602, define fluid chambers 718, 720, 722, and 724.

In the example of FIG. 7, body 602 includes fluid passages 726 and 728 between corresponding openings 614 and 616 and fluid chamber 724, a fluid passage 730 between fluid chamber 724 and opening 620 of assay region 618, and fluid passages between each of openings 622, 624, and 626 of assay region 618 and a waste chamber 740. Waste chamber 740 may include an absorptive material to receive fluid from one or more fluid chambers of system 600. Body 602 may include a fluid passage 742 between waste chamber 740 and the outer surface of body 602, such as to release air displaced by fluid received within waste chamber 740.

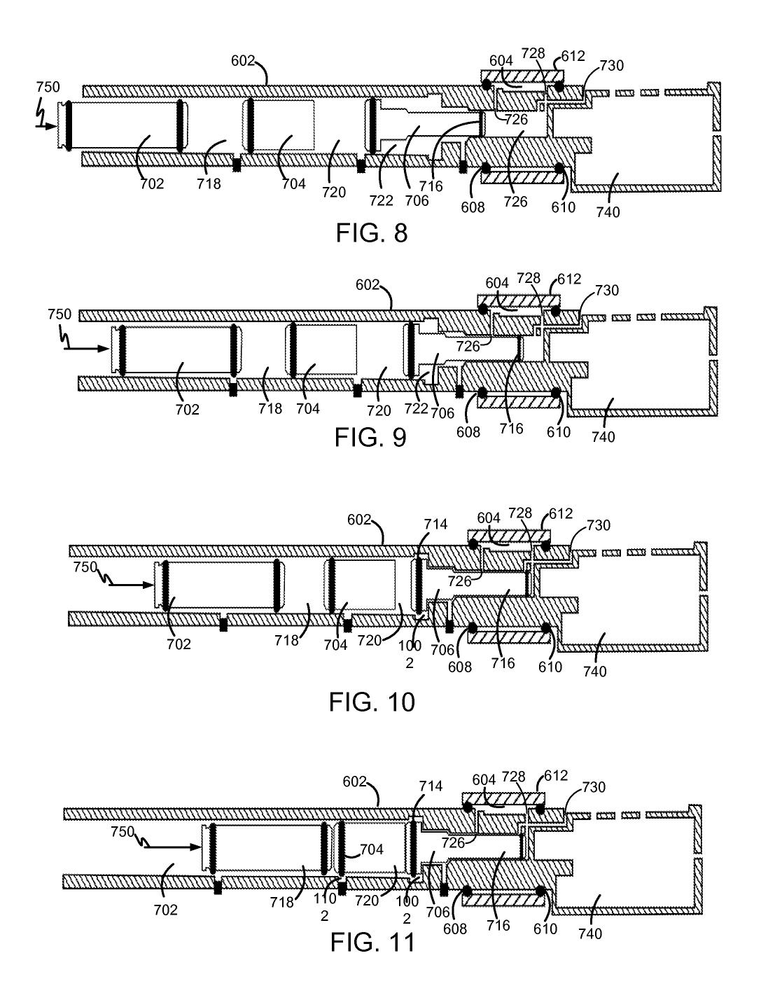

Body 602 may include one or more of fluid passages 744, 746, and 748 in fluid communication with corresponding fluid chambers 718, 720, and 722. One or more of fluid passages 744, 746, and 748 may have an opening through the outer surface of body 602, which may be used to provide one or more assay fluids to a corresponding fluid chamber during preparation procedure. Such an opening through the outer surface of body 602 may be plugged or sealed subsequent to the preparation procedure, such as illustrated in FIGS. 8-11. Alternatively, or additionally, one or more of fluid passages 744, 746, and 748 may include an opening to another fluid chamber of system 600, such as to provide a fluid bypass around one or more other fluid chambers and/or plungers.

Example operation of system 600 is described below with reference to FIGS. 8-14.

FIG. 8 is a cross-sectional view of system 600, wherein plungers 702, 704, and 706 are in corresponding initial or first positions.

FIG. 9 is a cross-sectional view of system 600, wherein plungers 702, 704, and 706 are in respective first intermediate positions.

FIG. 10 is a cross-sectional view of system 600, wherein plunger 704 is in a second position, and plungers 702 and 704 are in respective second intermediate positions.

FIG. 11 is a cross-sectional view of system 600, wherein plungers 702, 704 and 706 are in respective second positions.

FIGS. 8-11 may represent sequential positioning of plungers 702, 704 and 706 in response to a force in a direction 750 of FIG. 7.

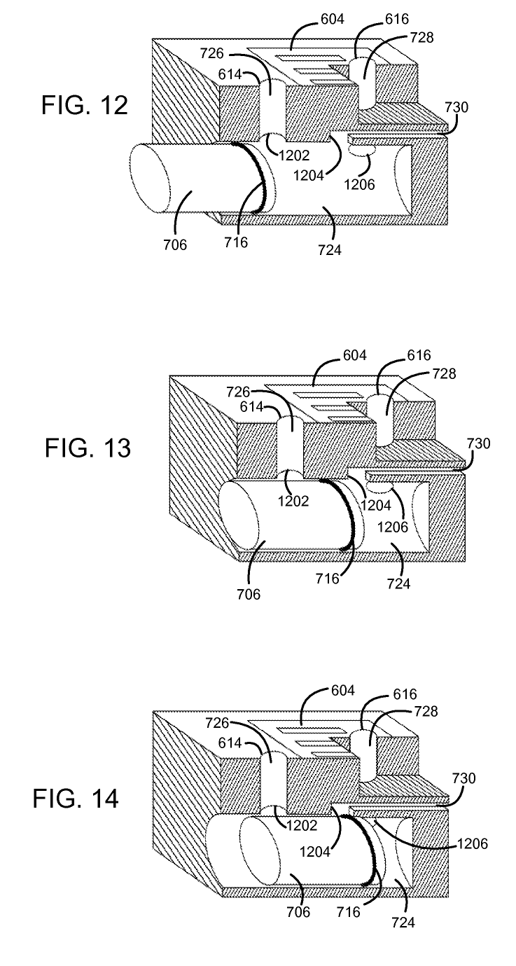

FIG. 12 is an expanded view of a portion of system 600, including a portion of plunger 706 in the first position corresponding to FIG. 8.

FIG. 13 is an expanded view of a of portion system 600, including a portion of plunger 706 in the intermediate position corresponding to FIG. 9, and including fluid directional arrows.

FIG. 14 is an expanded view of a portion of system 600, including a portion of plunger 706 in the second position corresponding to FIGS. 10 and 11.

During a preparation process, fluid chambers 718, 720, and 722, may be provided with corresponding first, second, and third fluids, and fluid chamber 724 may provided with a gas, such as air. The fluids in one or more of fluid chambers 718, 720, and 722 may be relatively incompressible compared with the gas in fluid chamber 724.

In FIGS. 8, when the force is applied to plunger 702 in direction 750, the relatively incompressibility of the fluids in fluid chambers 718 and 720 transfer the force to plunger 706. Plungers 702, 704, and 706 may move together in direction 750.

As plungers 702, 704, and 706 move in direction 750, fluid within fluid chamber 724, which may include air, travels from fluid chamber 724, through fluid passage 730 to assay chamber 732, and through fluid passages 734, 736, and 738 to waste chamber 740.

Prior to O-ring 716 of plunger 706 passing an opening 1202 (FIG. 12) of fluid passage 726, fluid chamber 722 is substantially isolated and no fluid flows from fluid chamber 722 to fluid channel 728 or from fluid chamber 722 to fluid chamber 724.

As O-ring 716 of plunger 706 moves towards opening 1202, and as fluid chamber 722 is correspondingly moved in direction 750 into a narrower-diameter inner surface portion of body 602, a volume of fluid chamber 722 decreases. The reduced volume of fluid chamber 722 may increase a pressure of the fluid within fluid chamber 722. The fluid within fluid chamber 722 may include a combination of a relatively incompressible fluid and relatively compressible fluid, such as air, which may compress in response to the increased pressure.

In FIG. 9, when O-ring 716 is positioned between opening 1202 of fluid passage 726 and an opening 1204 of fluid passage 730, fluid chamber 722 is in fluid communication with fluid channel 726, while O-ring 716 precludes fluid flow directly between fluid chambers 722 and 724. The fluid in fluid chamber 722 may thus travel from fluid chamber 722, through fluid passage 726 to sample collection region 604, through fluid passage 728 to fluid chamber 724, through fluid passage fluid passage 730 to assay region 618, and through openings 722, 724, and 726 to waste chamber 740.

The fluid from fluid chamber 722 may contact and dislodge at least a portion of a sample contained within a sample pad 606, and may carry the sample to assay region 618, where the sample may react with a test membrane.

In FIGS. 10, as plunger 706 reaches the second position and O-ring 716 passes opening 1204, a recess 1002 within an inner surface of body 602 provides a fluid passage around O-ring 714. Fluid within fluid chamber 720 travels through recess 1002, alongside plunger 706, through fluid passage 730 to assay chamber 732, and through fluid passages 734, 736, and 738 to waste chamber 740.

In FIGS. 11, as plunger 704 reaches the second position, a recess 1102 within an inner surface of body 602 provides a fluid passage around O-ring 712 of plunger 704. Recess 1102 may correspond to fluid channel 746 in FIG. 7. Fluid within fluid chamber 718 travels through recess 1102, alongside plunger 704, through recess 102, alongside plunger 706, through fluid passage 730 to assay chamber 732, and through fluid passages 734, 736, and 738 to waste chamber 740.

As illustrated in FIG. 14, when plunger 706 is in the second position, O-ring 716 may be positioned between an opening 1402 of fluid channel 728 and an opening 1404 of fluid channel 730 to preclude fluid flow from sample collection region 604 to assay chamber 732 through fluid channels 728 and 730. This may be useful, for example, where the fluids within fluid chamber 720 and 718 are to contact an assay membrane within assay chamber 732 rather than sample pad 606 within sample collection region 604. This may be useful, for example, where the fluids within fluid chamber 720 and 718 include a wash fluid and/or a reactive material to wash and/or react with the assay membrane.

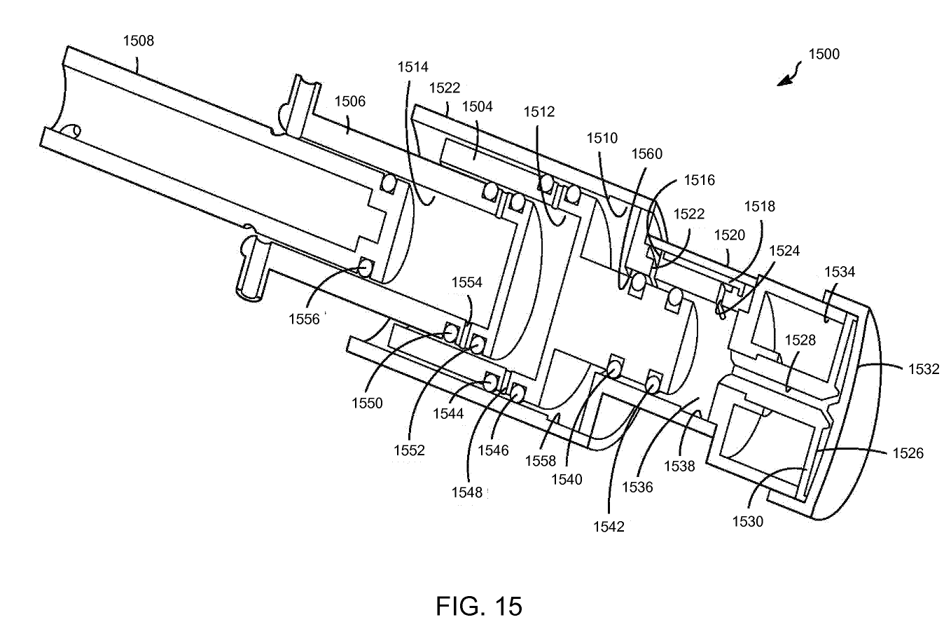

FIG. 15 is a cross-sectional perspective view of a portion of an assay system 1500 including a housing portion 1502 and a fluid controller system, including a plurality of fluid controllers, or plungers 1504, 1506, and 1508. Fluid controllers 1504, 1506, and 1508 define a plurality of fluid chambers, illustrated here as first, second, and third fluid chambers 1510, 1512, and 1514, respectively. Fluid controllers 1504, 1506, and 1508 are slideably nested within one another.

Housing portion 1502 includes a sample chamber 1516 to receive a sample, and may include a sample substrate, membrane or pad 1518. Housing portion 1502 may include a cover mechanism such as a cover portion 1520, which may be removable or hingedly coupled to housing portion 1502, as described above with respect to FIG. 3. Housing portion 1502 includes a sample chamber inlet 1522 and a sample chamber outlet 1524.

Housing portion 1502 includes an assay chamber 1526 and an assay chamber inlet 1528, and may include an assay substrate, membrane or pad 1528 to capture, react, and/or display assay results.

Housing portion 1502 includes an assay result viewer, illustrated here as a display window 1532 disposed over assay chamber 1528.

Housing portion 1502 includes a waste fluid chamber 1534 to receive fluids from assay chamber 1526.

Housing portion 1502 includes a transient fluid chamber 1536 having one or more fluid channels 1538, also referred to herein as a fluid controller bypass channel.

Housing portion 1502 further includes one or more other fluid channels 1558.

First fluid chamber 1510 includes a fluid chamber outlet 1560, illustrated here as a space between fluid controller 1506 and an inner surface of hosing portion 1502.

Second fluid chamber 1512 includes a fluid chamber outlet 1548, illustrated here as a gate or passage through fluid controller 1504.

Third fluid chamber 1514 includes a fluid chamber outlet 1554, illustrated here as a gate through fluid controller 1506.

Fluid controllers 1504, 1506, and 1508 include one or more sealing mechanisms, illustrated here as O-rings 1540 and 1542, O-rings 1544 and 1546, O-rings 1550 and 1552, and O-ring 1556.

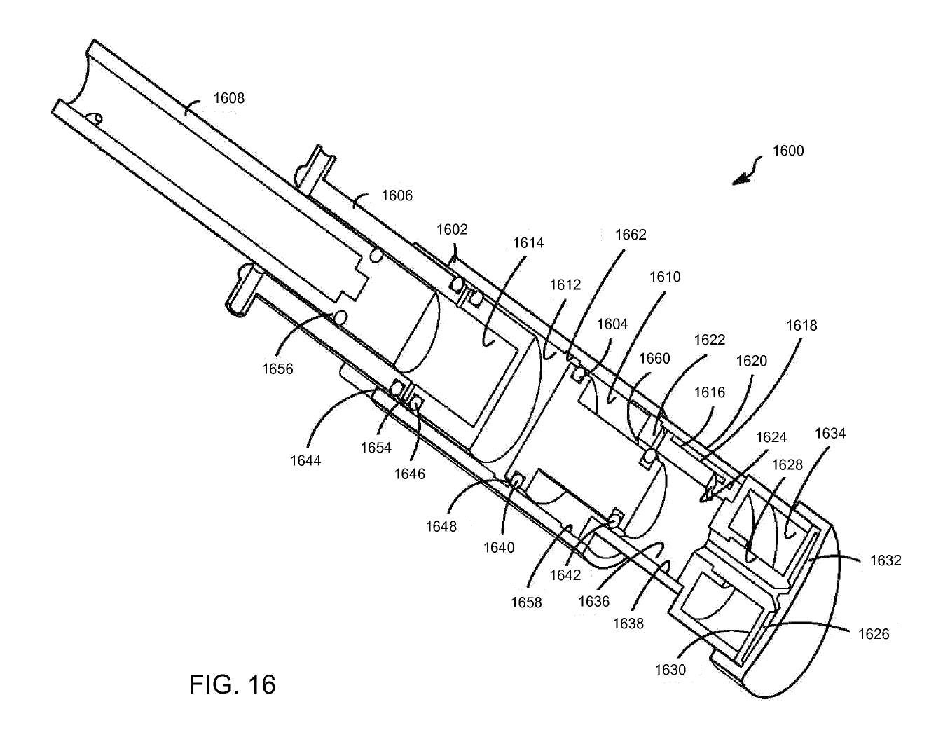

FIG. 16 is a cross-sectional perspective view of a portion of an assay system 1600 including a housing portion 1602 and a fluid controller system, including a plurality of fluid controllers, or plungers 1604, 1606, and 1608. Fluid controllers 1604, 1606, and 1608 define a plurality of fluid chambers, illustrated here as first, second, and third fluid chambers 1610, 1612, and 1614, respectively. Fluid controller 1608 is slideably nested within fluid controller 1606.

Housing portion 1602 includes a sample chamber 1616 to receive a sample, and may include a sample substrate 1618, which may include a surface of sample chamber 1616 or membrane therein. Housing portion 1602 may include a cover mechanism such as a cover portion 1620, which may be removable or hingedly coupled to housing portion 1602, as described above with respect to FIG. 3. Housing portion 1602 includes a sample chamber inlet 1622 and a sample chamber outlet 1624.

Housing portion 1602 includes an assay chamber 1626 and an assay chamber inlet 1628, and may include an assay substrate 1628 to capture, react, and/or display assay results. Assay substrate may include a surface of assay chamber 1626 or a membrane therein.

Housing portion 1602 includes an assay result viewer, illustrated here as a display window 1632 disposed over assay chamber 1628.

Housing portion 1602 includes a waste fluid chamber 1634 to receive fluids from assay chamber 1626.

Housing portion 1602 includes a transient fluid chamber 1636 having one or more fluid channels 1638, also referred to herein as a fluid controller bypass channel.

Housing portion 1602 further includes fluid channels 1658 and 1662.

First fluid chamber 1610 includes a fluid chamber outlet 1660, illustrated here as a space between fluid controller 1606 and an inner surface of hosing portion 1602.

Second fluid chamber 1612 includes a fluid chamber outlet 1648, illustrated here as a space between fluid controller 1604 and an inner surface of hosing portion 1602.

Third fluid chamber 1614 includes a fluid chamber outlet 1654, illustrated here as a gate or passage through fluid controller 1606.

Fluid controllers 1604, 1606, and 1608 include one or more sealing mechanisms, illustrated here as O-rings 1640 and 1642, O-rings 1644 and 1646, and O-ring 1656.

One or more inlets, outlets, openings, channels, and fluid pathways as described herein may be implemented as one or more of gates and passageways as described in one or more preceding examples, an may include one or more of:

a fluid channel within an inner surface of a housing;

a fluid passage within a housing, having a plurality of openings through an inner surface of the housing;

the fluid passage through a fluid controller; and

a fluid channel formed within an outer surface of one of the fluid controllers.

One or more inlets, outlets, openings, channels, fluid paths, gates, and passageways, as described herein, may include one or more flow restrictors, such as check valves, which may include a frangible check valve, to inhibit fluid flow when a pressure difference across the flow restrictor valve is below a threshold.

In FIG. 2, user-initiated actuator 204 may include one or more of a mechanical actuator, an electrical actuator, an electro-mechanical actuator, and a chemical reaction initiated actuator. User-initiated actuator systems are disclosed below, one or more of which may be implemented with one or more pumps disclosed above.

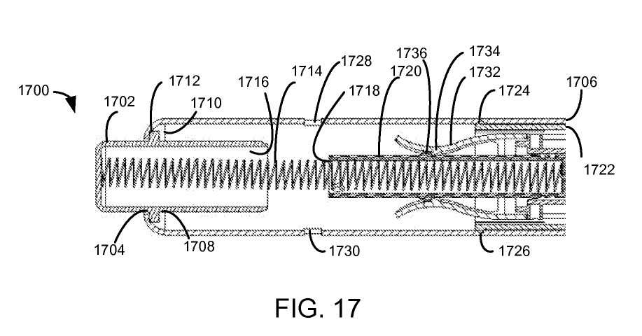

FIG. 17 is cross-sectional view of a mechanical actuator system 1700. Actuator system 1700 includes a button 1702 slideably disposed through an opening 1704 of an outer housing portion 1706, and through an opening 1708 of a frangible inner wall 1710 of outer housing portion 1706. Button 1702 includes a detent 1712 that extends beyond openings 1704 and 1708 to secure button 1702 between housing portion 1706 and frangible inner wall 1710.

Actuator system 1700 includes a compressible spring 1714 having a first end positioned within a cavity 1716 of button 1702, and a second end disposed within a cavity 1718 of a member 1720. Member 1720 may be coupled to, or may be a part of a fluid controller system, such a part of a plunger or fluid controller as described and illustrated in one or more examples herein.

Actuator system 1700 includes an inner housing portion 1722, slideably engaged within outer housing portion 1706. Inner housing portion 1722 includes one or more detents, illustrated here as detents 1724 and 1726, to lockingly engage one or more corresponding openings 1728 and 1730 in an inner surface of outer housing portion 1702.

Actuator system 1700 includes one or more frangible snaps 1732 coupled, directly or indirectly, to inner housing portion 1722. Frangible snap 1732 includes a locking detent 1734, and member 1720 includes a corresponding locking detent 1736 to releasably couple member 1720 to frangible snap 1732.

An assay system as disclosed herein may include a user-rupturable membrane to separate a plurality of chemicals within a flexible tear-resistant membrane. The chemicals may be selected such that, when combined, a pressurized fluid is generated. The pressurized fluid may be gas or liquid. The pressurized fluid may cause fluid controllers to move as described in one or more examples above. Multiple user-rupturable membranes may be implemented for multiple fluid passages.

Methods and systems to trap or capture bubbles are disclosed below.

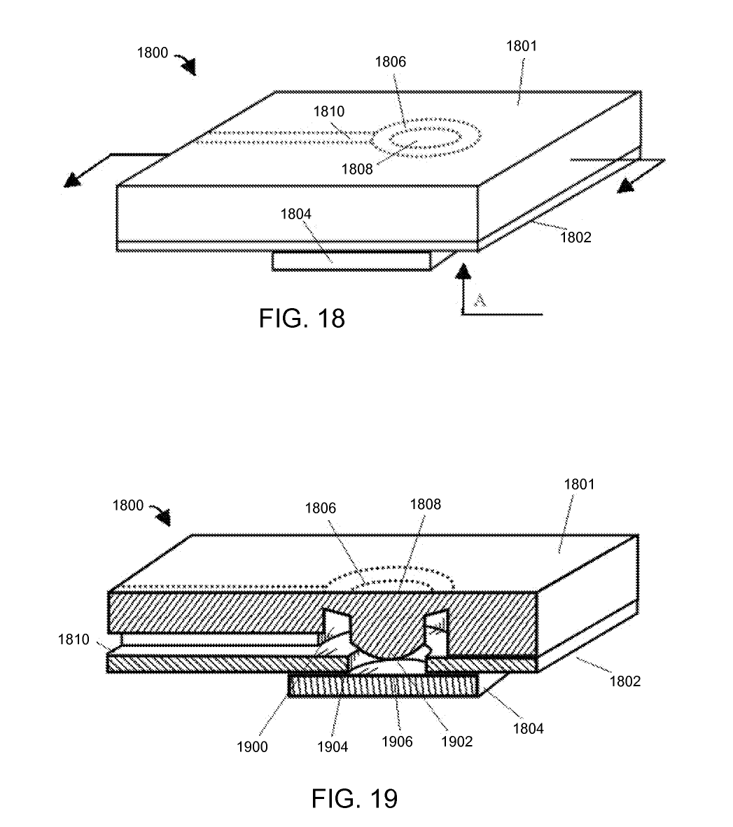

FIG. 18 is a profile view of a bubble trap system 1800.

FIG. 19 is a cross-sectional view of bubble trap system 1800.

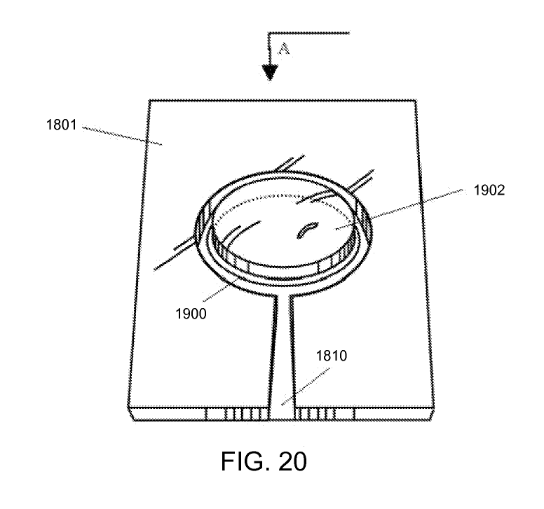

FIG. 20 is an upwardly directed view of an upper portion 1801 of bubble trap system 1800.

In FIG. 19, system 1800 includes a fluid channel 1810 to provide fluid to an opening or orifice 1904 through a surface of system 1800.

System 1800 may include a porous membrane 1804 positioned over orifice 1904 to receive fluid from fluid channel 1810. Porous membrane 1804 may include an active region, which may coincide with orifice 1904, and which may include a substance immobilized thereon. The substance may include, for example, an element to participate in a binding reaction, such as to detect the presence of a binding partner in a fluid sample.

System 1800 further includes a bubble termination pathway 1806 to receive, capture, or trap gas bubbles from fluid that flows through fluid channel 1810 to orifice 1904. Bubble termination pathway 1806, or a portion thereof, may be located vertically higher that at least a portion of fluid channel 1810 to permit gas bubbles to rise upwardly from fluid channel 1810. Gas bubbles may remain within bubble termination pathway 1806 due to buoyancy.

Bubble termination pathway 1806 may include a cavity 1900 (FIG. 19), having dimensions to hold a predetermined amount or volume of gas bubbles.

System 1800 may include a core portion 1808 having a lower surface 1902 disposed above orifice 1904 and defining a cavity 1906 therebetween. Lower surface 1902 may be substantially convex, which may assist in directing gas bubbles from cavity 1906, orifice 1904, and/or porous membrane 1804, toward cavity 1900, such as in response to gravity and/or centrifugal force.

Bubble termination pathway 1806, cavity 1900, core 1808, orifice 1904, and/or cavity 1906 may be in substantially vertical alignment with one another. Bubble termination pathway 1806, cavity 1900, core 1808, orifice 1904, and/or cavity 1906 may have substantially annular shapes, and may be in annular alignment with one another. Cavity 1900 may substantially encircle core 1808.

Bubble termination pathway 1806 may include a slanted upper surface, which may encourage distribution of gas bubbles throughout bubble termination pathway 1806.

Bubble termination pathway 1806, or a portion thereof, may be positioned outside of a circumference of orifice 1904, which may provide improved separation of gas bubbles from fluid, and which may provide an increased volume of space to hold or trap gas bubbles. permit increased.

Fluid channel 1810 may be in substantially horizontal alignment with a surface of core portion 1808, which may assist in separating gas bubbles from fluid, and which may assist in trapping gas bubbles in bubble termination pathway 1806.

System 1800 may include an upper portion 1801 and a lower portion 1802, which may be sealed together such as by adhesion, chemical solvents, or mechanical force (such as ultrasonics).

Upper portion 1801, or portions thereof, may be implemented with, for example, a substantially rigid clear material, such as a plastic, which may include one or more of styrene, polystyrene, nylon, polycarbonate or other suitable material.

Lower portion 1802, or portions thereof, may be implemented with, for example, a relative thin polystyrene material.

Porous membrane 1804 may be implemented with, for example, a nitrous cellulose membrane, and lower portion 1802 may be implemented with a material that can seal to a nitrous cellulose membrane 1804.

Bubble termination pathway 1806 and/or cavity 1900 may be sized to accommodate a predetermined, expected, or anticipated amount of gas to be trapped.

Orifice 1904 and/or an active area of porous membrane 1804 may be sized to expose a desired amount of membrane 1804 to accommodate the surface area of the active region to be in contact with a fluid. Orifice 1904 and/or an active area of porous membrane 1804 have a diameter of, for example, approximately 0.125 inches, which may provide for suitable involvement with the active region of the membrane although it will be readily recognized by those skilled in the art that many dimensions may be suitable depending on the assay to be performed, the strength of the detectable signal desired and the sensitivity to be achieved.

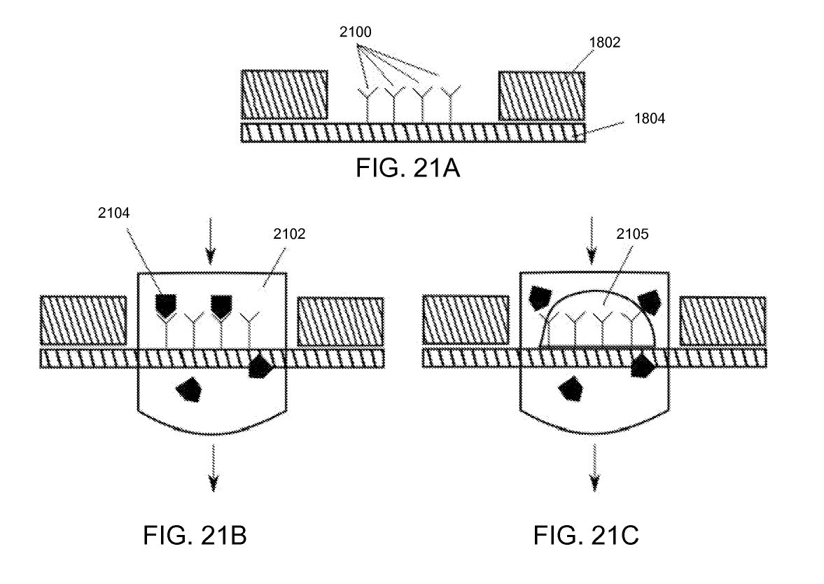

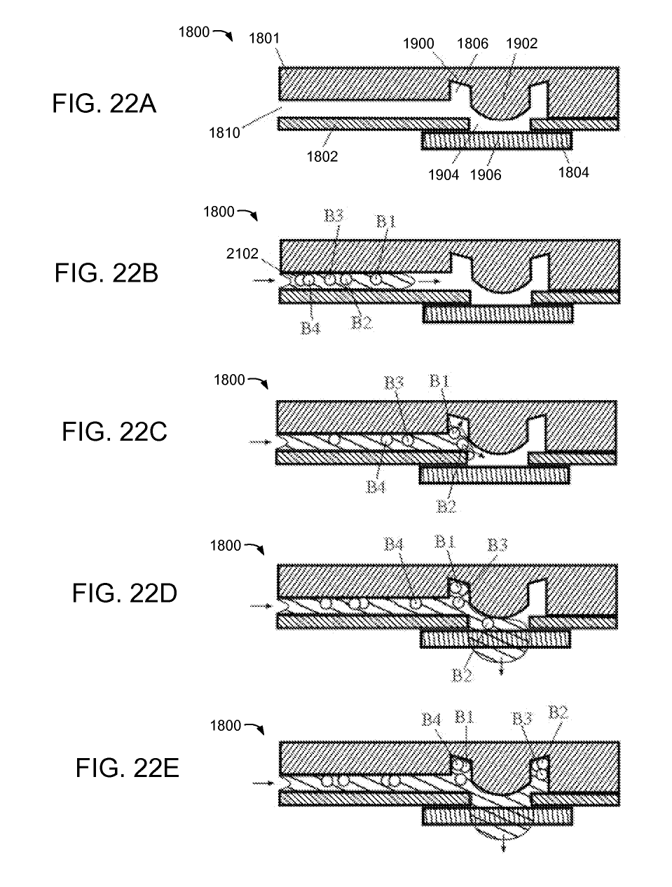

Example operation of system 1800 is described below with respect to FIG. 21A through 21C and FIGS. 22A through 22E.

FIG. 21 depicts movement of fluid that may be a fluid sample, regent fluid or a combination thereof and may contain gaseous bubbles. The active area of the membrane 1804 may contain markers, 2100, that may bind to substances, 2104, in the liquid, 2102, shown in FIG. 21B. The fluid with these substances flow through the membrane and some of them may be captured by the markers. If a gas bubble, 2105, shown in FIG. 21C stays in contact with the membrane, it prevents access of these substances to the markers. Fluid will still flow through the membrane by going around the gas bubble, but the active region may not have full contact.

FIGS. 22A through 22E illustrate example operation of membrane bubble trap system 1800.

In FIGS. 22A through 22C, an active region of membrane 1804, where fluid is to pass through, is positioned over orifice 1904.

Membrane bubble trap system 1800 may be oriented such that upper portion 1801 is opposite to a gravitational pull or centrifugal force, and is substantially level, relative to FIGS. 22A through 22D, such that bubbles travel to bubble termination pathway 1806 by buoyancy forces, and fluid flows downwardly through membrane 1804, such as by gravitational force, centrifugal force, and/or fluid pressure.

In FIG. 22B, fluid 2100 is enters fluid channel 1810. Fluid 2100 may include a fluid sample, reagent fluid, or combination thereof, and may contain gaseous bubbles. For purposes of the instant explanation, four bubbles are depicted and labeled B1, B2, B3, and B4.

In FIG. 22C, when fluid 2100 reaches bubble termination pathway 1806, bubble B1 travels upwardly into the slanted portion of cavity 1900, and bubble B2 is shown as having been forced into cavity 1906, such as by a force of fluid 2100.

In FIG. 22D, bubble B2 may contact membrane 1804, and lower surface 1902 of core portion 1808 may redirect bubble B2 upwardly into bubble termination pathway 1806, as shown in FIG. 22E.

Also in FIG. 22E, bubble B3 has been pushed, relative to FIG. 22D, around bubble termination pathway 1806 to a position opposite bubbles B1 and B4.

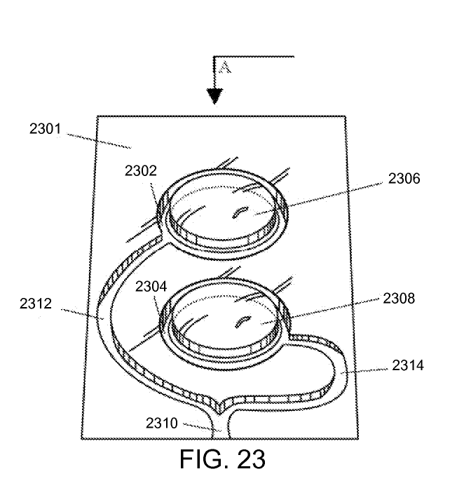

Bubble trap system 1800 may include multiple interconnected membrane active areas, each including a corresponding bubble termination trap. FIG. 23 illustrates an upper portion 2301 including multiple cavities 2302 and 2304 and corresponding curved sections 2306 and 2308.

Upper portion 2301 further includes a fluid channel 2310, including branches 2312 and 2314 to cavities 2302 and 2304.

Branches 2312 and 2314 may have similar fluid resistances and may be of similar length to permit fluid to reach corresponding active areas substantially simultaneously. More than one branch can end at the same bubble termination area.

Bubble trap system 1800 may be implemented within an assay system, such as one or more of assay systems 600, 1500, and 1600. For example, and without limitation, bubble trap system 1800 may be implemented to trap bubbles in an area proximate to a test membrane within assay region 618 in FIG. 6, wherein membrane 1804 of bubble trap system 1800 may be positioned over openings 622, 624, and 626 of assay region 618 in FIG. 6, and upper portion 1801 and lower portion 1802 of bubble trap system 1800, or portions thereof, may be implemented as part of body 602 and/or as part of a cover over assay region 618 of assay system 600. Fluid channel 110 of bubble trap system 100 may correspond to, or extend from fluid passage 730 of assay system 600 in FIG. 7.

While various embodiments are disclosed herein, it should be understood that they have been presented by way of example only, and not limitation. It will be apparent to persons skilled in the relevant art that various changes in form and detail may be made therein without departing from the spirit and scope of the methods and systems disclosed herein. Thus, the breadth and scope of the claims should not be limited by any of the example embodiments disclosed herein.

What is claimed is:

1. A structure to trap a predetermined amount of moving gas bubbles in a liquid, comprising:
a housing including one or more fluid pathways of predetermined cross-sectional shape and size connecting a source of fluid and an orifice having a predetermined size,
said orifice in fluid communication with a porous membrane,
a bubble collection pathway of predetermined cross-sectional shape and size surrounding said orifice and a central core and in fluid communication with said fluid pathways,
said central core aligned with said orifice and extending below said bubble collection pathway a predetermined distance, but not in contact with said porous membrane,
wherein said orifice and said central core cooperate to direct fluid flow onto said membrane while permitting gas bubbles to flow into and be collected by said bubble collection pathway.

2. The structure of claim 1 wherein said central core comprise optically transparent material selected from the group consisting of nylon, styrene, polystyrene, and polycarbonate.

3. A system, comprising:
a housing including a cavity therein, a first opening from the cavity through a lower surface of the housing, and a second opening from the cavity to a fluid channel to permit a fluid to flow from the fluid channel to the cavity, wherein the fluid channel includes an upwardly directed opening to a bubble pathway to permit bubbles in the fluid to rise into the bubble pathway.

4. The system of claim 3, further including:
a porous material sealing disposed against the lower surface of the housing and over the opening through the lower surface of the housing.

5. The system of claim 3, wherein the housing includes a convex portion disposed over the cavity to cause bubbles in the cavity to rise to the bubble pathway.

6. A method, comprising:
forcing a fluid into a fluid channel of a housing, wherein the housing includes a cavity, a first opening from the cavity through a lower surface of the housing, and a second opening from the cavity to the fluid channel to permit the fluid to flow from the fluid channel to the cavity, and wherein the fluid channel includes an upwardly directed opening to a bubble pathway to permit bubbles in the fluid to rise into the bubble pathway;
trapping gas bubbles from the fluid in the bubble pathway; and
passing the fluid through the opening in the lower surface of the housing.

7. The method of claim 6, wherein a porous material is sealing disposed against the lower surface of the housing and over the opening through the lower surface of the housing, the method further including:
passing the fluid through the porous membrane.

8. A system, comprising:
means for providing a fluid to a cavity of a housing; and
means for trapping gas bubbles from the fluid prior to the bubbles entering the cavity

9. The system of claim 8, further including:
means for directing gas bubbles within the cavity to the means for trapping.

10. The system of claim 8, wherein the housing includes an opening from the cavity through a lower surface of the housing, the system further including:
a porous material sealing disposed against the lower surface of the housing and over the opening through the lower surface of the housing.
